(19) United States
(12) Patent Application Publication
(10) Pub. No.: US 2026/0309612 A1
(43) Pub. Date: Oct. 8, 2026

(54) BIOLOGICAL MEASUREMENT APPARATUS

(71) Applicant: CYTOMOS LIMITED, Edinburgh (GB)

(72) Inventors: Thomas CLAYTON, Edinburgh (GB); Alexander LABOHM, Edinburgh (GB)

(21) Appl. No.: 19/482,053

(22) PCT Filed: May 10, 2024
(86) PCT No.: PCT/GB2024/051222

§ 371 Date: Nov. 6, 2025

(30) Foreign Application Priority Data
May 11, 2023 (GB) 2307017.0

(51) Int. Cl. G01N 15/1031; G01N 15/01

(52) U.S. Cl. CPC G01N 15/1031; G01N 15/01

(57) ABSTRACT

A biological measurement apparatus (10) is disclosed that comprises a fluid passageway (34) for a fluid medium and any biological particles therein, a first stimulus electrode (102a) and a first sensing electrode (202a) providing a first measurement capacitor, operative through a first part of the fluid passageway (34), and a second stimulus electrode (102b) and a second sensing electrode (202b) providing a second measurement capacitor, operative through a second part of the fluid passageway (34). The first sensing electrode (202a) is electrically connected to the second sensing electrode (202b), which electrical connection is insulated from the flow passageway. A stimulation apparatus (12) is electrically coupled to the stimulus electrodes (102a, 102b) and simultaneously applies first and second electric stimulus signals to the first and second stimulus electrodes (102a, 102b) respectively, the first and second electric stimulus signals having the same form and opposite polarity, and a sensing circuit (13) has an input coupled to the electrical connection between the first and second sensing electrodes (202a, 202b).

The present invention relates to biological measurement apparatus.

The analysis of physical or chemical characteristics of biological particles finds use in the diagnosis of disease, in research, in clinical trials of drugs, and the like. It is known to employ measurement apparatus to carry out such analysis on an automatic basis with the apparatus being configured to provide for simultaneous multi-parametric measurement of particles. Typically, the biological particles of interest are suspended in a fluid before being introduced to the measurement apparatus where the suspended biological particles are subject to excitation and the response to the excitation is detected.

One example of such measurement apparatus utilises impedance or dielectric spectroscopy to measure characteristics of biological particles. Impedance or dielectric spectroscopy involves applying an electrodynamic field to a solution containing a biological particle and measuring the changes to the field due to the presence of the biological particle, eg caused by the complex electrical permittivity of a particle.

When a biological cell is measured by the measurement apparatus, the fluid medium surrounding the biological cell is also measured. The effect of measurement of the surrounding fluid medium is addressed by carrying out two measurements at the same time, namely a first measurement of a biological cell and fluid medium holding the biological cell, and a second measurement of fluid medium without a biological cell. The difference between the first and second measurements is then determined to provide a proper measurement of the biological cell. The measurement apparatus therefore comprises a first measurement component which makes the first measurement and a second measurement component which makes the second measurement, the first and second measurement components operative at the same time.

Where measurement involves applying a stimulus to a sample and measuring a response to the stimulus, such as in impedance or dielectric spectroscopy, the stimulus is usually of considerably greater amplitude than the amplitude of the response to the stimulus.

If the first and second measurements are misaligned in time, then parts of the stimuli are present in the difference between the first and second measurements with the parts of the stimuli considerably greater in amplitude than amplitude of responses to the stimuli. Furthermore, the parts of the stimuli occupy the available voltage range of processing electronics to much greater extent than the responses to the stimuli. There is thus reduction in utilisation of the available voltage range of the processing electronics by the responses to the stimuli.

The present invention has been devised in light of the problems discussed above.

There have now been devised improved biological measurement apparatus which overcome or substantially mitigate the aforementioned and/or other disadvantages associate with the prior art.

According to a first aspect of the present invention, there is provided biological measurement apparatus, the biological measurement apparatus comprising
a fluid passageway for a fluid medium and any biological particles therein;
a first stimulus electrode and a first sensing electrode providing a first measurement capacitor, operative through a first part of the fluid passageway or a first fluid passageway;
a second stimulus electrode and a second sensing electrode providing a second measurement capacitor, operative through a second part of the fluid passageway or a second fluid passageway;
the first sensing electrode being electrically connected to the second sensing electrode, which electrical connection is insulated from the flow passageway;
a stimulation apparatus electrically coupled to the stimulus electrodes and simultaneously applying first and second electric stimulus signals to the first and second stimulus electrodes respectively, the first and second electric stimulus signals having the same form and opposite polarity; and
a sensing circuit having an input coupled to the electrical connection between the first and second sensing electrodes, and an electric response signal generated at an output.

According to a further aspect of the present invention, there is provided a method of biological measurement, the method comprising:
providing a device as defined above,
simultaneously applying first and second electric stimulus signals to the first and second stimulus electrodes respectively, the first and second electric stimulus signals having the same form and opposite polarity; and
generating an electric response signal at the output of the sensing circuit.

Since the stimulation circuit simultaneously applies first and second stimulus signals to the first and second stimulus electrodes respectively, with the first and second stimulus signals being of the same form and of opposite polarity, the first and second measurement capacitors have common responses of opposite polarity to the first and second stimuli by way of the fluid medium that is common to their dielectrics. The common responses of opposite polarity result in a net zero signal at the electrical coupling between the first and second measurement capacitors. Where a biological particle in the fluid medium affects the dielectric of one of the measurement capacitors, the effect of the biological particle is thus present at the electrical coupling between the first and second measurement capacitors.

The present biological measurement apparatus therefore obviates the need for the two measurement circuits that is conventional in the prior art. Instead, there is one measurement circuit, which reduces the problems associated with a mismatch between the paths of two measurement circuits in a differential measurement. Further to this, one measurement path according to the invention means half the noise of the conventional approach which has two measurement paths. In addition, since the output signal in the present invention is a smaller signal associated with the effect of the biological particle, rather than the effect of the particle and the fluid medium in the prior art approach, means measurement of the response to the at least one biological particle and subsequent processing is much less affected by non-linearity in the measurement and processing path.

In addition, although previous attempts have been disclosed for providing an electrode array for a net zero measurement, this prior art has used a single sense electrode that is disposed between two stimulus electrodes. The inventors found that this electrode arrangement is very sensitive to the position of the cell relative to the sensing electrodes. In particular, the optimal position for the cell in that arrangement is equidistant from one of the stimulus electrodes and the sensing electrode, and also close to the surface of those electrodes. However, the electric response signal reduces dramatically in strength as the position of the cell moves away from this optimal position. For example, the electric response signal is zero when the cell is over the sensing electrode, and hence equidistant from the two stimulus electrodes. In view of the very small size of biological cells, there are significant challenges in respect of making this prior art feasible for biological cells.

The inventors have found that providing a sense electrode that is split, or separated, in the flow channel, but is electrically connected under an insulating layer, provides the same net zero arrangement as a single sensing electrode arrangement, but separates the electric fields generated by the two stimulation electrodes, such that the electric response signal is less sensitive to the position of the cell relative to the sensing electrodes. The present invention therefore provides a workable net zero measurement apparatus suitable for biological particles.

The use of this configuration of net zero measurement apparatus may provide advantages in other measurement apparatus, either for measuring particles within a fluid medium or differences in the fluid medium. Hence, according to a further aspect of the invention, there is provided measurement apparatus comprising
a fluid passageway for a fluid medium;
a first stimulus electrode and a first sensing electrode providing a first measurement capacitor, operative through a first part of the fluid passageway;
a second stimulus electrode and a second sensing electrode providing a second measurement capacitor, operative through a second part of the fluid passageway;
the first sensing electrode being electrically connected to the second sensing electrode, which electrical connection is insulated from the flow passageway;
a stimulation apparatus electrically coupled to the stimulus electrodes and simultaneously applying first and second electric stimulus signals to the first and second stimulus electrodes respectively, the first and second electric stimulus signals having the same form and opposite polarity; and
an output circuit having an input coupled to the electrical connection between the first and second sensing electrodes, and a measurement signal generated at an output from the buffer circuit.

According to a further aspect of the present invention, there is provided a method of measurement, the method comprising:
providing a device as defined above,
simultaneously applying first and second electric stimulus signals to the first and second stimulus electrodes respectively, the first and second electric stimulus signals having the same form and opposite polarity; and
generating an electric response signal at the output of the sensing circuit.

The measurement apparatus may be configured to provide the first measurement capacitor with fluid medium in the flow passageway, but no biological particle, as a complex dielectric between the first stimulus electrode and the first sensing electrode of the first measurement capacitor. The measurement apparatus may be configured to provide the second measurement capacitor with fluid medium and at least one biological particle in the flow passageway as a complex dielectric between the second stimulus electrode and the second sensing electrode of the second measurement capacitor.

The flow passageway may direct the fluid material and any particles therein over both the first and second measurement capacitors. If only one particle passes the first and second measurement capacitors at a time, the measurement apparatus may be configured to measure the particle whether it passes over the first measurement capacitor or the second measurement capacitor.

Alternatively, the measurement apparatus may be configured to direct fluid medium that does not contain particles over the first measurement capacitor and fluid medium that does contain particles over the second measurement capacitor. This may be achieved by applying a force to the particles in a single flow passageway, such that the particles are directed over the second capacitor, for example using hydrodynamic focusing, dielectrophoresis focusing or inertial focusing. The measurement apparatus may have two flow passageways, eg capillaries, a first flow passageway directing fluid medium not containing particles over the first measurement capacitor and a second flow passageway directing fluid medium containing particles over the second measurement capacitor.

As the first and second electric stimulus signals provided to the first and second stimulus electrodes respectively are identical, but have an opposite polarity, and the dielectric between the electrodes of each measurement capacitor is the same when no biological cell is present in the fluid medium, the response signals provided by the sensing electrodes to the electrical connection therebetween, and hence to the coupling with the output circuit, will cancel each other out and a net zero output will be obtained.

When a cell is located adjacent to one of the measurement capacitors, but there is no cell present adjacent to the other measurement capacitor, one of the measurement capacitors will have both a biological particle and the fluid medium as a dielectric, whereas the other measurement capacitor will have the fluid medium, but no biological particle, as a dielectric. The presence of the biological particle will cause the response signals provided by the sensing electrodes to the electrical connection therebetween, and hence to the coupling with the output circuit, to be different, such that a non-zero difference caused by the biological particle will be obtained as at output.

The sensing circuit may include a buffer circuit and may be configured to compare the signal obtained from the electrical connection between the first and second sensing electrodes with a reference signal. The output signal is processed to determine one or more properties of a biological cell sensed by the measurement capacitor.

The first and second measurement capacitors may have operative surfaces configured to electrically couple with the fluid medium and any particle in the flow passageway, eg such that the fluid medium and any particle in the flow passageway forms a dielectric for the first and second measurement capacitors.

The operative surfaces of the first and second measurement capacitors may be exposed to the fluid medium, with no intermediate layer. The operative surfaces of the first and second measurement capacitors may conduct into the fluid medium.

The electrodes may be generally in cuboidal, and the operative surfaces of the electrodes may be square or rectangular. The operative surfaces of the electrodes may be flat. The sensing electrodes and the stimulation electrodes may be provided on an exposed surface of a semiconductor, and may be formed in an integrated circuit formed by a semiconductor fabrication process, such as CMOS.

The operative surfaces of the first and second measurement capacitors may lie in substantially the same plane. Alternatively, the plates of the first and second measurement capacitors may each lie in a respective plane, the respective planes substantially parallel to one another.

The electrodes of the first measurement capacitor may be disposed on or towards a side of the flow passageway, such as in a direction perpendicular to a direction of flow of the fluid medium. The electrodes of the second measurement capacitor may be disposed on or towards a side of the flow passageway. The electrodes of the first and second measurement capacitors may be disposed on or towards substantially a same side of the flow passageway. The electrodes of each measurement capacitor may be disposed side by side. Such a same side disposition may be appropriate where the particle stimulation apparatus is comprised in a planar semiconductor integrated circuit, such as a CMOS integrated circuit.

Where stimulation electrode and the sensing electrode are arranged with their operative surfaces in a generally planar arrangement, side-by-side, the fluid medium and any biological particle therein may flow over the operative surfaces of the electrodes.

The electrodes of the first and second measurement capacitors may take the form of an electrode array, which is formed on a surface of a semiconductor device. The apparatus may comprise a plurality of electrode arrays, ie a plurality of pairs of first and second measurement capacitors. The electrodes may be formed by metal plates, which are exposed to the flow of fluid medium. The fabrication process for the electrode array may lack a polyimide layer deposition step, such that no polyimide top layer is present. The electrode array may have a passivation layer, eg a silicon nitride layer, but may be without any passivation layer over the electrodes, such that the electrodes conduct into the fluid medium. The hydrophilic nature of the passivation layer provides for maximum exposure, with the openings in the passivation layer between the electrodes and the fluid medium enabling conduction between the electrodes and the fluid medium. The size of the electrodes is selected depending on the size of the cells being measured.

The operative surfaces of the stimulus electrodes of the first and second measurement capacitors may be separated from each other, aligned transversely relative to the flow of fluid medium, eg with the first stimulus electrode being aligned with a flow of fluid medium only, ie without particles to be measured, and the second stimulus electrode may be aligned with a flow of fluid medium and particles to be measured.

The operative surfaces of the sensing electrodes of the first and second measurement capacitors may be separated from each other, aligned transversely relative to the flow of fluid medium, eg with the first stimulus electrode being aligned with a flow of fluid medium only, ie without particles to be measured, and the second stimulus electrode may be aligned with a flow of fluid medium and particles to be measured. The first and second electrodes are nevertheless electrically connected to each other, eg under an insulating layer, such as the passivation layer.

The sensing electrodes may each be disposed adjacent to, but separated from, a corresponding stimulus electrode, thereby providing the first and second measurement capacitors. In each measurement capacitor, the stimulus electrode may be upstream or downstream of the sensing electrode, and the first and second measurement capacitors may have the same arrangement.

An electrode may have a dimension, such as at least one of width and height, of less than substantially 100 microns, 50 microns, 30 microns, 20 microns, 15 microns, 10 microns, 5 microns, 3 microns or 1 micron. Alternatively or in addition, an electrode may have a dimension of more than substantially 0.5 microns, 1 micron, 5 microns, 10 microns, 15 microns, 20 microns, 30 microns or 50 microns.

The distance between the electrodes of the first measurement capacitor and the electrodes of the second measurement capacitor may be selected such that there is a sufficient separation of the respective electrical fields for a particle at one measurement capacitor to have a small or negligible impact on the response of the other measurement capacitor.

A second complementary net zero electrode arrangement may be provided, which may be disposed upstream or downstream of the net zero electrode arrangement described above. The electrodes of this complementary net zero electrode arrangement may have the same layout and circuitry as the electrodes of the first net zero electrode arrangement.

The same electric stimulus signal may be provided to the stimulus electrodes of each of the first and second net zero electrode arrangements, with the stimulus electrodes of the first measurement capacitor in each arrangement being electrically connected to each other and the stimulus electrodes of the second measurement capacitor in each arrangement being electrically connected to each other.

The output signals from the sensing circuit associated with the upstream and downstream net zero electrode arrangements may be compared, eg by subtraction, such that a differential measurement is made. In particular, the concentration of biological particles and the flow rate of the fluid medium may be controlled such that each biological particle in the fluid medium passes over one of the measurement capacitors of the first net zero electrode arrangement and then, subsequently, passes over one of the measurement capacitors of the second net zero electrode arrangement. The use of this differential arrangement enables any mismatch from sources affecting the measurement in each fluid stream to be removed. This mismatch may be in the rise and fall times of the stimulus, for example, or in the analyte, eg caused by the cells breathing and consequent changes to the fluid medium.

In one embodiment, the stimulus electrodes of the first measurement capacitor in each arrangement are combined, and provided by a single stimulus electrode, and the stimulus electrodes of the second measurement capacitor in each arrangement are combined, and provided by a single stimulus electrode. In this embodiment, the stimulus electrode of the first measurement capacitors of the net zero electrode arrangements may be disposed between the sensing electrodes of the first measurement capacitors of the net zero electrode arrangements, and the stimulus electrode of the second measurement capacitors of the net zero electrode arrangements may be disposed between the sensing electrodes of the second measurement capacitors of the net zero electrode arrangements.

In this arrangement, the stimulus electrodes act as an isolator between the first and second net zero electrode arrangements. An alternative option for providing isolation between the first and second net zero electrode arrangements, eg where the first and second net zero electrode arrangements have separate stimulus electrodes, would be to provide an electrode between the sensing electrodes of the first and second net zero electrode arrangements, with this electrode being in contact with the fluid medium and being provided with a DC voltage, such that the electric fields of the first and second net zero electrode arrangements are isolated from each other. However, this alternative arrangement uses a DC voltage which may have electrochemical effects that are undesirable. The use of combined stimulus electrodes of the first and second net zero electrode arrangements to provide isolation overcomes these disadvantages.

Furthermore, the use of combined stimulus electrodes of the first and second net zero electrode arrangements enables the distance between the two sensing electrodes in each fluid medium stream to be reduced relative to an arrangement in which the first and second net zero electrode arrangements have separate stimulus electrodes. This increases the maximum cell concentration that may be used with the apparatus, as the apparatus is controlled such that one cell crosses over the entire electrode array before a second cell starts to cross the electrode array.

The measurement apparatus may comprise, or be adapted to be operative with, a flow apparatus that provides for flow of the fluent material and the biological particles in the flow passageway. In use, a sample of fluent material, such as a biological sample, is introduced into a flow apparatus with the flow apparatus being configured to contain and provide for flow of the fluent material through the flow passageway. The flow apparatus may, for example, define an open-ended channel which contains the fluent material and allows for flow such as when flow is created by way of a pump. Alternatively or in addition, the flow apparatus may be configured to actuate flow of itself. More specifically the flow apparatus may be configured to draw the fluent material through the flow apparatus by way of capillary action.

The measurement apparatus may comprise an integrated circuit formed by a semiconductor fabrication process, such as a CMOS fabrication process. The particles may be biological particles, e.g. cells, cell clusters or other structures including viruses, multi-cell organisms, bacteria and spores. Sensing of particles may provide for analysis of the particles involving, for example, differentiation of cell species from each or one another.

The measurement apparatus may be configured to measure a quantity impacted by electrical permittivity of a particle. The measurement apparatus may be configured to measure a quantity impacted by at least one of real and imaginary electrical permittivity of a particle. The quantity being measured may be the change to an applied electrodynamic field, ie a stimulus signal, caused by the presence of a particle.

A particle, such as a cell, may interact with the applied electrodynamic field in a characteristic fashion so as to provide a response that may be measured. The measurement apparatus may therefore be used to measure a characteristic of a biological particle in fluent material. Furthermore, different biological particles may cooperate with the applied electrodynamic field in different characteristic fashions so as to provide corresponding different responses. The measurement apparatus may therefore be used to characterise a biological particle. For example, the measurement apparatus may be used to at least one of differentiate one type of cell from another type of cell and determine a characteristic of a cell, such as a dimension of the cell or a composition of a cell.

The apparatus according to the invention may also be configured to measure differences or changes in a fluid medium, eg a gradient across the fluid passageway. This may be achieved when no particles are present in the fluid passageway.

Alternatively, the apparatus according to the invention may be configured to measure differences or changes in a fluid medium when particles are present in the fluid passageway. In particular, the response obtained from a biological particle may be affected by changes to the fluid medium. Certain aspects of the response, eg at particular frequencies or frequency ranges, may be dominated by a change in the fluid medium. Hence, if the output changes, the change could be due to a change in the biological particles being measured or a change in the fluid medium, or both.

Changes to the biological particle and/or fluid medium may be detected by changes to the distribution of responses over time, which may indicate a change in the biological particles being measured or a change in the fluid medium, or both. For example, if a change to the output is detected, but this change is uniform across cells, this may indicate that the change is due to changes in the fluid medium. Alternatively, if there is a gradual change in the output over time and there is increased variance between biological particles, this may indicate that the change is due to changes in the biological particles. Machine learning techniques may be used to interpret these changes.

Hence, in a bioreactor, the apparatus according to the invention may be configured to determine if changes in the output are due to changes in the fluid medium or changes in the biological cells, eg increasing cell heterogeneity, which may be determined using machine learning techniques, for example.

The sensing circuit may comprise a high impedance input to thereby provide for a significant sensed signal. More specifically the sensing circuit may comprise an impedance buffer, such as a field-effect transistor (FET). A FET may provide a capacitive load, e.g. for a sensing electrode of the sensing apparatus, which may be selected to be as small as possible. Alternatively or in addition, the sensing circuit may provide one of a voltage signal and a current signal as the output signal.

Alternatively or in addition, the sensing circuit may be configured to amplify the input signal. The measurement apparatus may be configured to compare the electric stimulus signal with the electric response signal, e.g. as sensed by the sensing apparatus. Comparison of the electric stimulus signal with the electric response signal may comprise cross-correlation of the electric stimulus signal with the electric response signal. The measurement apparatus may be operative to determine a time delay between application of the electric stimulus signal and the response as provided by at least one particle in the fluent material. The measurement apparatus may be further operative to determine a transfer function or at least to approximate a transfer function for the at least one particle in dependence on the time delay. The determined or approximated transfer function may then provide for characterisation of the at least one particle.

The pre-determined, electrodynamic field applied to the biological particle may comprise at least one corresponding frequency component. The sensing apparatus may be configured to sense a frequency component comprised in the response electrodynamic field, sensed by the particle sensing apparatus, the frequency component being at least 1 kHz, 10 kHz, 50 kHz, 100 kHz, 250 kHz, 500 kHz, 1 MHz, 5 MHz, 10 MHz, 25 MHz, 50 MHz, 75 MHz, 100 MHz, 250 MHz, 500 MHz, 750 MHz, 1 GHz, 1.25 GHz, 1.5 GHz, 1.75 GHz, 2 GHz. 2.5 GHz, 2.75 GHz or 5 GHz. More specifically the frequency component may be between 10 kHz and 5 GHz, between 1 kHz and 10 GHz or between 1 kHz and 20 GHz. Under certain circumstances, at a frequency above 1 MHz the measurement apparatus may be operative to characterise an inside of the at least one particle, such as where the at least one particle is a biological cell. Under certain circumstances, a frequency above 1 kHz may be required to characterise a particle in respect of its external characteristics, such as a size of a biological cell.

The present invention is particularly advantageous when used as biological measurement apparatus in Process Analytics Technology (PAT), as it may enable continual monitoring of cell cultures whilst they are within a bioreactor and may therefore enable more reliable and/or more detailed data to be obtained relative to prior art monitoring systems.

The electric stimulus signal may comprise a pseudo-random binary sequence having a length and data rate that provide the pre-determined, electrodynamic field with a range of frequencies. The measurement apparatus may be configured to generate the pseudo-random noise signal. The pseudo-random noise signal may be generated from a pseudo-random binary sequence.

The pseudo-random binary sequences are generated with a deterministic algorithm. The pseudo-random binary sequence nevertheless exhibits statistical behaviour similar to a truly random sequence. The pseudo-random binary sequence may be a maximum length sequence, a so-called “m-sequence”, which may be generated by a linear feedback shift register. Where a binary sequence is generated by a linear feedback shift register, the output eventually repeats itself. An m-sequence is the longest possible non-repeating sequence for a given number of shift registers.

The pseudo-random noise signal may therefore comprise an m-sequence. M-sequences exhibit a flat power spectral density across a desired bandwidth of operation. Furthermore, m-sequences may be readily provided for by way of standard digital circuitry and thus may be suited to implementation in an integrated circuit formed by a semiconductor fabrication process, such as CMOS.

The stimulation circuit may be operative to generate a stimulation signal in the form of an m-sequence by way of a linear feedback shift register or otherwise as would be within the ordinary design skills of the person skilled in the art. For example, the stimulation circuit may comprise memory storing the m-sequences. Alternatively, an m-sequence may be provided by an external signal generator.

The length of each m-sequence is 2ⁿ−1, where n is the number of registers in the linear-feedback shift register. The range of frequencies provided by the pseudo-random binary sequence may be determined by the length of each of the pseudo-random binary sequence, determined in numbers of bits, and the bit rate of the electric stimulus signal.

The range of frequencies of the pseudo-random binary sequence is determined by the binary signal achievable with the length of the pseudo-random binary sequence and the bit rate of the electric stimulus signal. The highest frequency of the range of frequencies may be an alternating sequence of bits, immediately adjacent to each other, which will have a frequency of half of the bit rate. The lowest frequency may be a constant value across the entire length of the sequence, which will have a frequency of the bit rate divided by the length of the sequence.

An n-register m-sequence will have a length of 2ⁿ−1 bits. At a data rate of f, the n-register m-sequence will provide frequencies of f/2 to f/(2ⁿ−1). For example, a 5-register m-sequence will have a length of 31 bits. At a data rate of 950 MHz, the 5-register m-sequence will provide frequencies of 475 MHz to 30.65 MHz. In contrast, a 7-register m-sequence will have a length of 127 bits. At a data rate of 50 MHz, the 7-register m-sequence will provide frequencies of 25 MHz to 393 kHz.

The measurement apparatus may comprise processing apparatus, which may include the stimulation circuit and/or the sensing circuit. The processing apparatus and/or the sensing circuit may be configured to receive the electric response signal and to convert the electric response signal to digital form. The processing apparatus and/or the sensing circuit may therefore have an analogue input and a digital output, such that the output is a digital response signal.

The processing apparatus and/or the sensing circuit may therefore comprise an analogue-to-digital converter and whatever signal conditioning circuitry may be required, such as an amplifier and an anti-aliasing filter. The processing apparatus may be constituted by any suitable electronic apparatus, such as a separate analogue-to-digital converter circuit, a separate amplifier circuit and a separate filter circuit or a configurable integrated circuit, such as an FPGA, or a dedicated integrated circuit, such as an Application Specific Integrated Circuit (ASIC), comprising such circuits.

The same clock may be used for the generation of the electric stimulus signal and the sample rate of the processing apparatus. This ensures that the electric stimulus signal does not drift in time relative to the digital response signal. The sample rate of the processing apparatus may, however, be different to the bit rate of the electric stimulus signal, as discussed in more detail below. The sample rate of the processing apparatus and the bit rate of the electric stimulus signal may also be expressed in terms of the sample period of the processing apparatus and the bit period of the electric stimulus signal. Where the clock used for the generation of the electric stimulus signal and the clock used for sampling are different, the respective clocks are preferably derived from the same clock.

The digital response signal may be stored in memory. The processing apparatus and/or the sensing circuit may comprise a processor and may be operative to decode the received output signal, which may be m-sequence encoded data.

The impulse response may be calculated by application of a Hadamard transform, such as the Fast Hadamard transform. This provides for rapid calculation of the impulse response. The fast Hadamard transform is given by:

$${Ψ}^{′} = \frac{1}{{2}^{m - 1}} {ξ}_{1} H {ξ}_{2} η$$

where Ψ is the estimated output spectrum of the system under test, m is the sequence order, H is the Hadamard matrix, η is the measured m-sequence encoded response, and ξ₁ and ξ₂ are the encode and decode matrices for transforming m-sequence data into the correct order for use with the Hadamard matrix. In one form, ξ₁ and ξ₂ are equal to each other.

Alternatively, analysis apparatus may be configured to decode the received output signal, which may be m-sequence encoded data, for example by cross-correlation. The analysis apparatus may be constituted by any suitable electronic apparatus, e.g. a general purpose computer, such as a Personal Computer (PC).

The analysis apparatus may be further operative to perform a Fourier Transform, such as a Fast Fourier Transform (FFT), on the decoded output signal to thereby provide frequency domain data. The frequency domain data may be displayed for user interpretation. The frequency domain data may provide for characterisation of the biological cells in the analyte, such as in respect of their dimensions and composition whereby the nature of a particular cell type can be determined or different cell types can be differentiated.

The measurement apparatus may be operative and perhaps also configured to be label free. The biological sensing apparatus may therefore operate on fluent material lacking any label, such as a fluorochrome or microbeads.

The measurement apparatus may be operative and perhaps also configured for sensing of microbiological samples. The measurement apparatus may be configured for a particular size of particle or a range of sizes of particles in respect of a dimension of at least one of the stimulation apparatus and the sensing apparatus, such as a size of at least one electrode. More specifically, a dimension of at least one of the stimulation apparatus and the sensing apparatus may correspond to a size of particle or a range of sizes of particles.

The semiconductor fabrication process may be a planar semiconductor fabrication process. Alternatively or in addition, the semiconductor fabrication process may be a metal-oxide semiconductor process, such as a CMOS process. Alternatively or in addition, the semiconductor fabrication process may be a submicron semiconductor fabrication process, such as a 0.18 micron CMOS process and perhaps a high voltage 0.18 micron CMOS process.

The fluent material may be substantially liquid, e.g. at room temperature. The fluent material may therefore comprise a liquid which carries the particles. More specifically, the fluent material may comprise charge carriers, such as salt molecules. For example the fluent material may comprise phosphate buffered saline (PBS).

The flow apparatus may define a fluid passageway through which the fluent material flows when in use. A single fluid passageway may be provided for the first and second measurement capacitors, or a fluid passageway may be provided for each of the first and second measurement capacitors, ie first and second fluid passageways. In particular, the first and second fluid passageways may have cross-sectional dimensions that ensure that a particle to be measured flows through the operative sensing area of the corresponding measurement capacitor.

This fluid passageway configuration may also assist in ensuring that the particle flows in close proximity to the electrodes.

The sensing apparatus may be disposed relative to the fluid passageway so as to provide for sensing of particles present in the fluid passageway. The sensing apparatus may be disposed on at least one of first and second opposite sides of a flow of fluent material. Thus, for example, components such as sensing electrodes of the sensing apparatus may be disposed on one side of the flow of fluent material. According to another example, components of the particle sensing apparatus may be disposed on both sides of the flow of fluent material.

The flow apparatus may comprise a sample inlet which is configured to receive a sample of fluent material, e.g. by way of injection, which is to be subject to measurement, the sample inlet being in fluid communication with the main channel. The flow apparatus may comprise a sample outlet at an opposite end of the flow apparatus from the sample inlet, the sample outlet being in fluid communication with the main channel. The sample outlet may provide for flow of fluent material from the main channel.

The flow apparatus may comprise at least one further inlet disposed laterally of the sample inlet. More specifically, the flow apparatus may comprise first and second further inlets with the first inlet disposed laterally on one side of the sample inlet and the second inlet disposed laterally on another opposite side of the sample inlet. The at least one further inlet may be in fluid communication with the main channel. In use, a sheath fluid, such as phosphate buffered saline (PBS), may be received by the at least one further inlet to thereby provide for a flow of sheath fluid in the main channel, the flow of sheath fluid being lateral of a flow of fluent material. The flow of sheath fluid may provide for registration of the particle comprising fluent material with the particle sensing apparatus and may also help preserve the integrity of the flow of fluent material as it progresses though the flow apparatus.

The flow apparatus may be formed from glass and/or at least in part from a polymer, such as poly(methyl methacrylate) (PMMA). The stimulation and sensing apparatus may be formed separately from the flow apparatus. The stimulation and sensing apparatus may be disposed relative to the flow apparatus by attaching the stimulation and sensing apparatus and the flow apparatus to each other. The stimulation and sensing apparatus may comprise co-planar stimulation and sensing electrodes and surrounding substrate, eg with a substantially planar exposed surface, over which the flow apparatus is disposed, such that the main channel through which the fluent material flows is defined between the co-planar electrodes and the flow apparatus. The flow apparatus may be insulating and may be devoid of electrodes or electrically conductive material. The stimulation and sensing apparatus and the flow apparatus may be adhered together, for example by appropriate chemical or physical bonding, or may be mechanically attached to each other, e.g. by way of a fastener apparatus comprising a silicone gasket layer, which may be releasable. Appropriate chemical or physical bonding may include plasma bonding.

The biological measurement apparatus may be configured to be operable as a flow cytometer. The measurement apparatus may further comprise control apparatus. The control apparatus may be constituted by any suitable electronic apparatus, such as a microprocessor or a configurable electronic circuit, such as a Field Programmable Gate Array (FPGA).

The measurement apparatus may comprise flow inducing apparatus, i.e. a pump, which is operative to induce a flow of fluent material through the flow apparatus. The flow inducing apparatus may be controlled, for example in respect of a rate of flow of fluent material through the flow apparatus, in dependence on an output from the sensing apparatus. The control apparatus may be operative to receive an output from the sensing apparatus and to provide an output to the flow inducing apparatus in dependence thereon.

The sensing apparatus as described elsewhere herein may be operative to provide for determination of a rate of flow of fluent material through the flow apparatus, the rate of flow being received by the control apparatus. Where the sensing apparatus comprises plural spaced apart sensing electrodes with each sensing electrode being operative to sense particles, the rate of flow may be determined in dependence on the separation between the sensing electrodes being known and a time between sensing of a particle by different sensing electrodes.

Alternatively or in addition, characterisation of at least one particle as described elsewhere herein may be compared with a predetermined criterion and the flow inducing apparatus may be controlled in dependence on the comparison. For example, characterisation of the at least one particle may comprise a level of confidence value which is compared with a predefined value. More specifically, if the level of confidence value is below the predefined value the flow inducing apparatus may be operative to reduce a rate of flow of the fluent material to thereby provide for improved characterisation.

The processing apparatus may provide an output signal, eg a digital output signal. The output signal may be stored in memory of the measurement apparatus. The measurement apparatus may comprise an output for sending the output signal to analysis apparatus, which may or may not be integrated with the measurement apparatus.

The analysis apparatus may be configured to make determinations with regards to particles comprised in the fluent material in dependence on at least one output from the particle sensing apparatus. For example, the analysis apparatus may be operative to make determinations in dependence on electric field measurements made by the particle sensing apparatus after analogue to digital conversion. Determinations may be made in respect of the like of the density of particles comprised in the fluent material, differentiation of one form of particle from another and characteristics of particles, such as in respect of dimensions or composition. The analysis apparatus may be constituted by any suitable electronic apparatus, e.g. a general purpose computer, such as a Personal Computer (PC), an embedded microprocessor, a configurable electronic circuit, such as a FPGA or the like. Further embodiments of the second aspect of the present invention may comprise one or more further features of the first aspect of the present invention.

The sensor chip may be configured to be mounted within a bioreactor, eg from microbioreactors to large bioreactors. Microbioreactors are particularly suitable for use in personalised medicine, eg small batches may be required on a per patient basis.

Further features and advantages of the present invention will become apparent from the following specific description, which is given by way of example only and with reference to the accompanying drawings, in which:

FIG. 1 is a block diagram representation of biological measurement apparatus according to a first embodiment of the present invention;

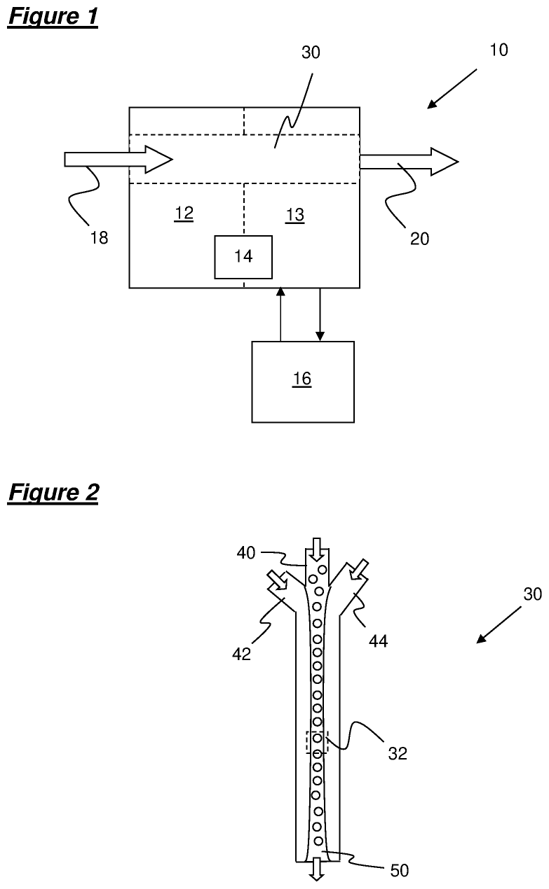

FIG. 2 is a representation of a flow apparatus that is part of the first embodiment of the biological measurement apparatus;

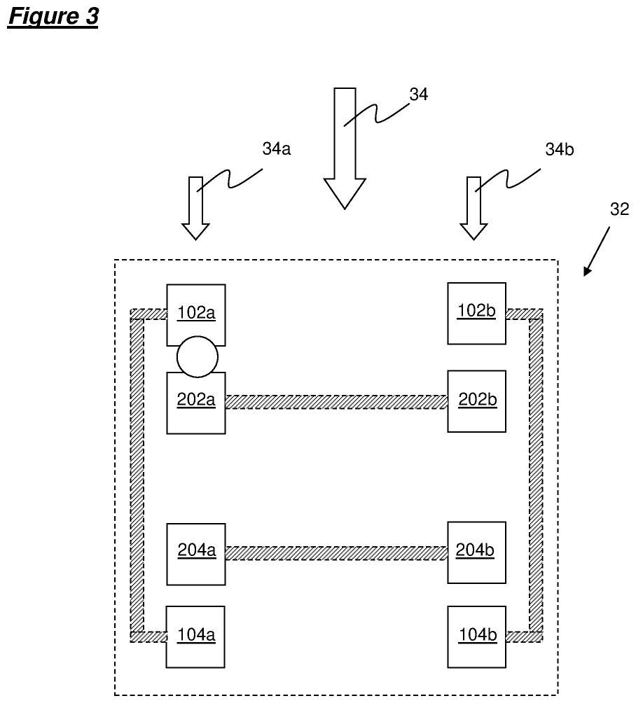

FIG. 3 is a representation of an electrode array that is part of the first embodiment of the biological measurement apparatus;

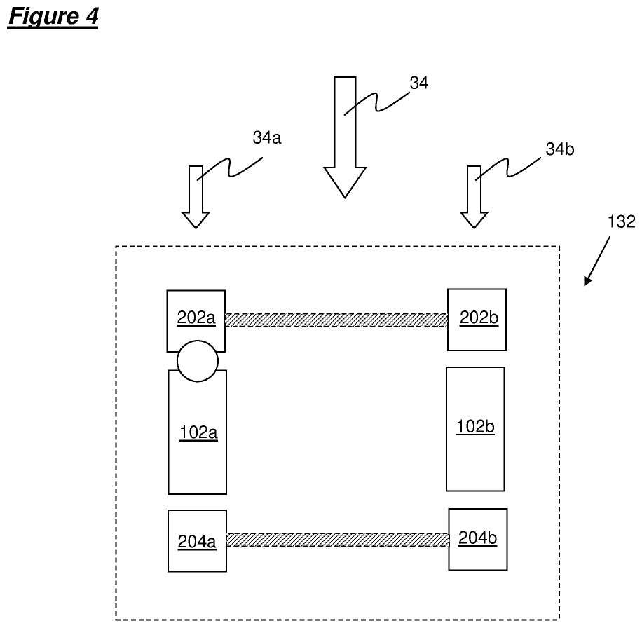

FIG. 4 is a representation of an electrode array that is part of the second embodiment of the biological measurement apparatus; and

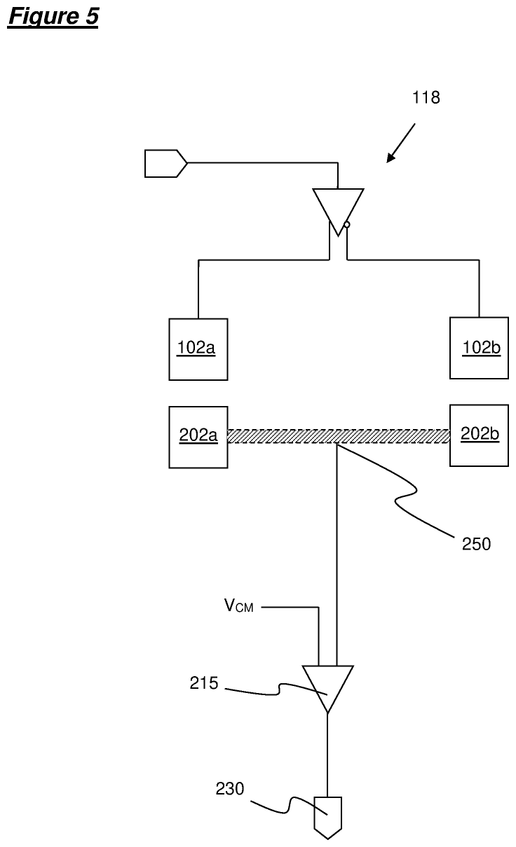

FIG. 5 is a circuit representation of sensing apparatus that is part of the first embodiment of the biological measurement apparatus.

A block diagram representation of biological measurement apparatus 10 according to the present invention is shown in FIG. 1. The biological analysis apparatus 10 comprises flow apparatus 30, stimulation apparatus 12, sensing apparatus 13, control and processing apparatus 14, and analysis apparatus 16. The stimulation apparatus 12 and sensing apparatus 13 receive, via the flow apparatus 30, a flow of analyte in the form of phosphate buffered saline (PBS) 18 (which constitutes fluent material) in which biological cells (which constitute particles) are suspended. An alternative to PBS may be used on account of the present approach providing for sensing which is independent of the suspension material.

The flow of analyte is directed by the flow apparatus 30 through the stimulation apparatus 12 and sensing apparatus 13 where it is subject to stimulation and sensing, as described in detail below, before exiting 20 from the measurement apparatus 10.

The control and processing apparatus 14 controls the application of stimulation signals to the analyte by the stimulation apparatus 12, and processes signals sensed by the sensing apparatus 13. Processing comprises amplification of sensed signals, analogue to digital conversion of sensed signals and storage of converted sensed signals. Although not shown in FIG. 1, the measurement apparatus 10 further comprises a pump which is operative to push or draw analyte through the stimulation apparatus 12 and sensing apparatus 13, via the flow apparatus 30.

The analysis apparatus 16 is operative to make at least one analytical determination in dependence on the stored converted sensed signals. Analytical determinations comprise: detecting the presence of biological cells in the analyte; counting biological cells in the analyte; differentiating one form of biological cell from another; and determining a characteristic of biological cells in the analyte, such as a cell dimension or a cell composition. The analysis apparatus 16 is also operative to provide for supervisory control of the control and processing apparatus 14, e.g. in respect of a change in the form of control of the biological sensing apparatus 12 exercised by the control and processing apparatus 14.

The control and processing apparatus 14 is constituted by any suitable electronic apparatus, such as a separate analogue-to-digital converter circuit, a separate amplifier circuit and a separate electronic memory circuit, or a configurable integrated circuit, such as a System-on-a-Chip (SOC), comprising the digital circuits and an ASIC comprising the analogue circuits. The analysis apparatus 16 is constituted by any suitable electronic apparatus, e.g. a general purpose computer, such as a PC, an embedded microprocessor, a configurable electronic circuit, such as an FPGA, or the like. The control and processing apparatus 14 and analysis apparatus 16 are constituted apart from each other, e.g. as separate modules, or constituted together, e.g. in a same integrated circuit or same general purpose computer.

The flow apparatus 30 receives the analyte 18 and provides for flow of the analyte before the analyte exits 20 from the flow apparatus. With reference to FIG. 2, the measurement apparatus also comprises a two-dimensional array of electrodes 32, which comprise stimulation electrodes of the stimulation apparatus 12 and sensing electrodes of the sensing apparatus 13. The flow apparatus 30 and the array of electrodes 32 are disposed in relation to each other such that the array of electrodes 32 is below a main channel of the flow apparatus 30.

The control and processing apparatus 14 is electrically coupled to the array of electrodes 32. The control and processing apparatus 14 is operative to provide for biological cell stimulation, sensing and actuation by way of the array of electrodes 32.

The arrangement of the array of electrodes is shown in more detail in FIG. 3.

The array of electrodes 32 are constituted by a CMOS process such as a 0.35 micron CMOS process. The array of electrodes 32 and the control and processing apparatus 14 are both comprised in a CMOS ASIC.

The thickness and permittivity of the standard polyimide top layer of the ASIC provides insufficient capacitance for proper engagement of the electrodes 32 with the analyte, and hence the fabrication process lacks a polyimide layer deposition step so no polyimide top layer is present. The hydrophilic nature of the silicon nitride layer provides for maximum exposure. However, the silicon nitride layer is removed over the electrodes, such that the electrodes conduct into the analyte. The ASIC is disposed, as is mentioned above, relative to the flow apparatus 30 such that the array of electrodes 32 engages with the analyte flowing through the flow apparatus 30.

The measurement apparatus 10 further comprises a Printed Circuit Board (PCB) which supports and provides for electrical connectivity for electrical circuits which support the ASIC. The electrical circuits comprised in the PCB includes an SOC which is configured to provide various digital functions including the generation of stimulus signals and communication with a Universal Serial Bus (USB) module.

The SOC 44 is operative to generate a stimulation signal in the form of an m-sequence that is stored in memory and output one bit at a time. The PCB also includes input signal conditioning circuitry which is configured to receive stimulus signals from the SOC or from the external (un-illustrated) signal generator. In addition, the PCB includes output signal conditioning circuitry which performs a variety of functions including fixed gain, low distortion amplification of sensed single ended signals followed by programmable gain amplification or attenuation of such initially amplified signals under the control of a PC.

The USB module provides for communication with a PC running software operative to perform the functions of the analysis apparatus 16 of FIG. 1. More specifically, the PC is operative to configure the ASIC 42 and the circuits comprised in the PCB. In addition, the PC receives real-time sensed data or blocks of data which have been acquired and stored locally from the SOC.

The PC is operative to decode the received m-sequence encoded data by cross-correlation to provide the impulse response. The PC is further operative to perform a Fast Fourier Transform (FFT) on the decoded data to thereby provide frequency domain data. The frequency domain data is then displayed for user interpretation. The frequency domain data provides for characterisation of the biological cells in the analyte, such as in respect of their dimensions and composition whereby the nature of a particular cell type can be determined or different cell types can be differentiated.

The PC is also operative to count biological cells present in the analyte and to determine a density of cells present in the analyte in dependence on the flow rate and volume of the flow apparatus with the count and density information being displayed to the user.

A representation of a flow apparatus 30 comprised in the biological sensing apparatus of FIG. 2 is shown in detail in FIG. 2. The flow apparatus 30 of FIG. 2 is formed from glass and has a length of about 25 mm and a width of about 10 mm. The flow apparatus 30 comprises a main channel 34 through which the analyte flows. The array of electrodes 32 is disposed above the main channel 34 so that the electrodes 32 engage with the analyte as the analyte flows through the main channel.

As is described above, the array of electrodes 32 is comprised in a CMOS ASIC. The CMOS ASIC and the flow apparatus 30 are bonded to each other, such that a proper relative disposition of electrodes and main channel is achieved.

The flow apparatus 30 also comprises a sample inlet 40 which receives the analyte, e.g. by way of injection, and a sample outlet 50 at an opposite end of the flow apparatus from the sample inlet 40. The sample inlet 40 and the sample outlet 50 are each in fluid communication with the main channel 34. In addition, the flow apparatus comprises first and second further inlets 42, 44. The first further inlet 42 is disposed laterally on one side of the sample inlet 40 and the second further inlet 44 is disposed laterally on the other opposite side of the sample inlet. Each of the first and second further inlets 42, 44 are in fluid communication with the main channel 34.

In use, a sheath fluid, such as phosphate buffered saline (PBS), is received by each of the first and second further inlets 42, 44 to thereby provide for a flow of sheath fluid in the main channel, the flow of sheath fluid being lateral of a flow of analyte received by the sample inlet 40. The flow of sheath fluid provides for registration of the biological cell comprising analyte with the array of electrodes 32 and also helps preserve the integrity of the flow of analyte as it progresses though the main channel.

Stimulation and sensing comprises electric field stimulation and electric field sensing. A biological cell cooperates with the applied electric field whereby the electric field is disturbed. Different sizes of a particular type of biological cell will disturb an applied electric field in a different manner. In addition, different types of biological cell will disturb an applied electric field in a different manner. Sensing of the electric field in dependence on the disturbance can therefore provide for detection of the presence of biological cells, determination of relative sizes of cells and differentiation of different types of cell from each or one another.

The array of electrodes 32 is shown in FIG. 3. In the flow apparatus of FIG. 2, the main channel of the flow apparatus 30 has a width that is sufficient for the array of electrodes 32 to be disposed within the flow of analyte 34, with the cells being focussed into a cell and fluid medium stream 34a that flows over electrodes 102a, 202a, 204a and 104a, and a fluid medium stream 34b (with no cells) that flows over electrodes 102b, 202b, 204b and 104b. The electrodes 102a, 102b, 202a, 202b, 204a, 204b, 104a and 104b are generally central in the main channel of the flow apparatus, where there is laminar flow.

The focusing of the cells may be achieved by any known method of manipulating the position of cells, such as hydrodynamic focussing, dielectrophoresis focusing, or the use of capillaries, eg one for each component of the flow 34a and 34b.

The electrodes in the array 32 are formed by metal plates, which are exposed to the flow of analyte. As discussed above, the fabrication process for the electrode array 32 lacks a polyimide layer deposition step so no polyimide top layer is present. The hydrophilic nature of the silicon nitride layer provides for maximum exposure. However, the silicon nitride layer is removed over the electrodes, such that the electrodes conduct into the analyte.

The size of the electrodes is selected depending on the size of the cells being measured. In the electrode array 32 of FIG. 3, electrodes 102a and 102b are a first pair of stimulus electrodes, which are each provided with an electric stimulus signal, as discussed in more detail below with reference to FIG. 4. The first pair of stimulus electrodes 102a and 102b are separated from each other, aligned transversely across the flow of analyte, with a first stimulus electrode 102a being aligned with the cell and fluid medium stream 34a and the second stimulus electrode 102b being aligned with the fluid medium stream 34b.

Electrodes 202a and 202b are a first pair of sensing electrodes. The surfaces of the sensing electrodes 202a and 202b that are exposed to the analyte are separated from each other, aligned transversely across the flow of analyte, with a first sensing electrode 202a being aligned with the cell and fluid medium stream 34a and the second sensing electrode 202b being aligned with the fluid medium stream 34b. Although surfaces of the sensing electrodes 202a and 202b that are exposed to the analyte are separated from each other, the sensing electrodes 202a and 202b are electrically connected to each other under the silicon nitride layer.

The sensing electrodes 202a,202b are each disposed adjacent to, but separated from, a respective stimulus electrode 102a,102b, thereby providing two stimulus-sensing pairs of electrodes—a stimulus-sensing pair of electrodes 102a and 202a aligned with the cell and fluid medium stream 34a, and a stimulus-sensing pair of electrodes 102b and 202b aligned with the fluid medium stream 34b. In each stimulus-sensing pair of electrodes, 102a, 202a and 102b, 202b, the stimulus electrode 102a,102b is upstream from the sensing electrode 202a,202b.

Each stimulus-sensing pair of electrodes, 102a, 202a and 102b, 202b, forms two plates of a capacitor, with the fluid medium and any cell forming a dielectric between those plates. In this embodiment, the stimulus electrodes 102a,102b and the sensing electrodes 202a,202b each have a size of 20 μm×20 μm. The separation between the stimulus electrode 102a,102b and the sensing electrode 202a,202b in each stimulus-sensing pair is 4 μm, and the separation between the stimulus and sensing electrodes 102a,202a aligned with the cell and fluid medium stream 34a, and the stimulus and sensing electrodes 102b,202b aligned with the fluid medium stream 34b, is 100 μm.

With reference to FIG. 4, the electric stimulus signals provided to stimulus electrodes 102a and 102b via input 118 are identical, but have an opposite polarity. The electric response signal is received from a connection 250 equidistant from each of the sensing electrodes 202a,202b.

When no cell is present in the cell and fluid medium stream 34a, both stimulus-sensing pair of electrodes, 102a, 202a and 102b, 202b, will have the fluid medium, but no cell, as a dielectric. As the electric stimulus signals provided to stimulus electrodes 102a and 102b via input 118 are identical, but have an opposite polarity, and the dielectric between each stimulus-sensing pair of electrodes, 102a, 202a and 102b, 202b, is the same (both fluid medium only), the response signals provided by the sensing electrodes 202a,202b will cancel each other out and a net zero output will be obtained from the connection 250 equidistant from each of the sensing electrodes 202a,202b.

When a cell is located adjacent to the stimulus-sensing pair of electrodes 102a and 202a aligned with the cell and fluid medium stream 34a, this stimulus-sensing pair of electrodes 102a and 202a will have both a cell and the fluid medium as a dielectric, whereas the other stimulus-sensing pair of electrodes 102b and 202b will have the fluid medium, but no cell, as a dielectric. The presence of the cell will cause the response signals provided by the sensing electrodes 202a,202b to be different, such that the difference caused by the cell will be obtained as at output, relative to zero, from the connection 250 equidistant from each of the sensing electrodes 202a,202b

The output from the connection 250 equidistant from each of the sensing electrodes 202a,202b is compared to a common mode voltage, and the difference is output 230 as the electric response signal.

Previous attempts at providing an electrode array for a net zero measurement have used a single sense electrode that is disposed between two stimulus electrodes. However, the inventors found that this electrode arrangement is very sensitive to the position of the cell relative to the sensing electrodes. In particular, the optimal position for the cell in that arrangement is equidistant from one of the stimulus electrodes and the sensing electrode, and also close to the surface of those electrodes. However, the electric response signal reduces dramatically in strength as the position of the cell moves away from this optimal position. For example, the electric response signal is zero when the cell is over the sensing electrode, and hence equidistant from the two stimulus electrodes.

The inventors have found that providing a sense electrode that is split, or separated, in the flow channel, but is electrically connected under an insulating layer, provides the same net zero arrangement as a single sensing electrode arrangement, but separates the electric fields generated by the two stimulation electrodes, such that the electric response signal is less sensitive to the position of the cell relative to the sensing electrodes.

In the electrode array 32 of FIG. 3, a complementary, downstream net zero electrode arrangement is provided by electrodes 104a,104b,204a,204b, disposed downstream of the upstream net zero electrode arrangement provided by electrodes 102a,102b,202a,202b described above. The electrodes 104a,104b,204a,204b of this complementary, downstream net zero electrode arrangement has the same layout and circuitry as the electrodes 102a,102b,202a,202b of the upstream net zero electrode arrangement, with the only difference being that the sensing electrodes 204a,204b of the downstream net zero electrode arrangement are disposed upstream of the stimulation electrodes 104a,104b.

The same electric stimulus signal is provided to the stimulus electrodes of each of the upstream and downstream net zero electrode arrangements, with stimulus electrodes 102a and 104a being electrically connected to each other and stimulus electrodes 102b and 104b being electrically connected to each other.

The output signals from the sensing circuit associated with the upstream and downstream net zero electrode arrangements are compared, eg by subtraction, such that a differential measurement is made. In particular, the concentration of biological particles and the flow rate is controlled such that each biological particle in the flow passes over the first pair of electrodes 102a,202a and then the second pair of electrodes 104a,204a. The differential measurement will be zero until a biological particle passes over the first pair of electrodes 102a,202a or the second pair of electrodes 104a,204a, when a differential signal will be output.

The use of this differential arrangement enables any mismatch from sources affecting the measurement in each fluid stream 34a,34b to be removed. This mismatch may be in the rise and fall times of the stimulus, for example, or in the analyte, eg caused by the cells breathing and consequent changes to the fluid medium.

The distance between the first pair of electrodes 102a,202a and the second pair of electrodes 104a,204a is selected to be sufficient that the respective electrical fields are separated, such that a biological particle isn't detected by both pairs of electrodes at the same time.

The electrode array 132 of a second embodiment of the biological measurement apparatus is shown in FIG. 4. This embodiment has the same arrangement of sensing electrodes 202a,202b,204a,204b, but has a single pair of stimulus electrodes 102a,102b. A first stimulus electrode 102a is disposed between the sensing electrodes 202a,204a that are aligned with the cell and fluid medium stream 34a, and a second stimulus electrode 102b is disposed between the sensing electrodes 202b,204b that are aligned with the fluid medium stream 34b. In other words, the corresponding stimulus electrodes of the complementary net zero electrode arrangements of the first embodiment have been merged in the second embodiment, and located between the sensing electrodes of the complementary net zero electrode arrangements. The stimulus electrodes 102a,102b of the second embodiment have a greater length in the direction of low in the second embodiment, which is selected to be sufficient that the respective electrical fields are separated, such that a biological particle isn't detected by both pairs of electrodes at the same time. In this embodiment, the stimulus electrodes 102a,102b each have a size of 20 μm×40 μm.

The stimulus electrodes 102a,102b of the second embodiment act as an isolator between the first and second net zero electrode arrangements. An alternative option for providing isolation between the first and second net zero electrode arrangements, eg in the first embodiment, would be to provide an electrode between the sensing electrodes of the first embodiment, with this electrode being in contact with the fluid medium and being provided with a DC voltage, such that the electric fields of the first and second net zero electrode arrangements are isolated from each other. However, this alternative arrangement uses a DC voltage which may have electrochemical effects that are undesirable. The use of combined stimulus electrodes 102a,102b to provide isolation overcomes these disadvantages.

Furthermore, the use of combined stimulus electrodes 102a,102b enables the distance between the two sensing electrodes in each fluid medium stream 34a,34b, namely the distance between sensing electrodes 202a and 204a and the distance between sensing electrodes 202b and 204b to be reduced relative to the first embodiment. This increases the maximum cell concentration that may be used with the apparatus, as the apparatus is controlled such that one cell crosses over the entire electrode array before a second cell starts to cross the electrode array.

The pump of the measurement apparatus 10 of FIG. 1 is controlled in dependence on at least one of: a rate of flow of analyte through the measurement apparatus 10; and a level of confidence of characterisation of the analyte flowing through measurement apparatus 10. Considering rate of flow of analyte further, the separation between pairs of electrodes in the array is known and the time of travel of biological cells between pairs of electrodes is determined by the control and processing apparatus 14.

The control and processing apparatus 14 is then operative to determine the speed of movement of biological cells through the measurement apparatus 10. The control and processing apparatus 14 is then operative to control the pump in dependence on the determined speed. For example, if the determined speed is below a predetermined value the control and processing apparatus 14 is operative to increase the flow rate by controlling the pump. Considering level of confidence of characterisation of the analyte further, the control and processing apparatus 14 is operative to characterise biological cells and to determine a level of confidence of the characterisation. The control and processing apparatus 14 is further operative to compare the determined level of confidence with a predetermined level and then to control the pump in dependence thereon. If the determined level of confidence is below the predetermined level the control and processing apparatus 14 is operative to reduce the rate of flow by controlling the pump to thereby provide for improved characterisation of the biological cells.

1. A biological measurement apparatus, the biological measurement apparatus comprising
a fluid passageway for a fluid medium and any biological particles therein;
a first stimulus electrode and a first sensing electrode providing a first measurement capacitor, operative through a first part of the fluid passageway or a first fluid passageway;
a second stimulus electrode and a second sensing electrode providing a second measurement capacitor, operative through a second part of the fluid passageway or a second fluid passageway;
the first sensing electrode being electrically connected to the second sensing electrode, which electrical connection is insulated from the flow passageway;
a stimulation apparatus electrically coupled to the stimulus electrodes and simultaneously applying first and second electric stimulus signals to the first and second stimulus electrodes respectively, the first and second electric stimulus signals having the same form and opposite polarity; and
a sensing circuit having an input coupled to the electrical connection between the first and second sensing electrodes, and an electric response signal generated at an output.

2. A biological measurement apparatus as claimed in claim 1, wherein the measurement apparatus is configured to provide the first measurement capacitor with fluid medium in the flow passageway, but no biological particle, as a complex dielectric between the first stimulus electrode and the first sensing electrode of the first measurement capacitor, and the measurement apparatus is configured to provide the second measurement capacitor with fluid medium and at least one biological particle in the flow passageway as a complex dielectric between the second stimulus electrode and the second sensing electrode of the second measurement capacitor.

3. A biological measurement apparatus as claimed in claim 1 or claim 2, wherein the flow passageway directs the fluid medium and any particles therein over both the first and second measurement capacitors.

4. A biological measurement apparatus as claimed in claim 3, wherein the flow passageway directs the fluid medium and any particles therein over both the first and second measurement capacitors, such that only one particle passes the first and second measurement capacitors at a time.

5. A biological measurement apparatus as claimed in claim 1 or claim 2, wherein the measurement apparatus is configured to direct fluid medium that does not contain particles over the first measurement capacitor and fluid medium that does contain particles over the second measurement capacitor.

6. A biological measurement apparatus as claimed in claim 5, wherein the measurement apparatus has two flow passageways, a first flow passageway directing fluid medium not containing particles over the first measurement capacitor and a second flow passageway directing fluid medium containing particles over the second measurement capacitor.

7. A biological measurement apparatus as claimed in any preceding claim, wherein the sensing circuit includes a buffer circuit and is configured to compare the signal obtained from the electrical connection between the first and second sensing electrodes with a reference signal.

8. A biological measurement apparatus as claimed in any preceding claim, wherein the output signal is processed to determine one or more properties of a biological cell sensed by the measurement capacitor.

9. A biological measurement apparatus as claimed in any preceding claim, wherein the first and second measurement capacitors have operative surfaces configured to electrically couple with the fluid medium and any particle in the flow passageway, such that the fluid medium and any particle in the flow passageway forms a dielectric for the first and second measurement capacitors.

10. A biological measurement apparatus as claimed in claim 9, wherein the operative surfaces of the first and second measurement capacitors are exposed to the fluid medium, with no intermediate layer.

11. A biological measurement apparatus as claimed in claim 9 or claim 10, wherein the operative surfaces of the first and second measurement capacitors conduct into the fluid medium.

12. A biological measurement apparatus as claimed in any preceding claim, wherein the sensing electrodes and the stimulation electrodes are provided on an exposed surface of a semiconductor, and formed in an integrated circuit formed by a semiconductor fabrication process.

13. A biological measurement apparatus as claimed in any one of claims 9 to 12, wherein the operative surfaces of the first and second measurement capacitors lie in substantially the same plane, or lie in a respective plane, the respective planes substantially parallel to one another.

14. A biological measurement apparatus as claimed in any preceding claim, wherein the electrodes of the first measurement capacitor are disposed on or towards a side of the flow passageway, such as in a direction perpendicular to a direction of flow of the fluid medium, and the electrodes of the second measurement capacitor are disposed on or towards a side of the flow passageway.

15. A biological measurement apparatus as claimed in any preceding claim, wherein the electrodes of the first and second measurement capacitors are disposed on or towards substantially a same side of the flow passageway.

16. A biological measurement apparatus as claimed in any preceding claim, wherein the electrodes of each measurement capacitor are disposed side by side, such that the fluid medium and any biological particle therein flows over the operative surfaces of the electrodes.

17. A biological measurement apparatus as claimed in any preceding claim, wherein the electrodes of the first and second measurement capacitors may take the form of an electrode array, which is formed on a surface of a semiconductor device, and the electrode array may have a passivation layer, but without any passivation layer over the electrodes, such that the electrodes conduct into the fluid medium.

18. A biological measurement apparatus as claimed in any preceding claim, wherein the operative surfaces of the stimulus electrodes of the first and second measurement capacitors are separated from each other, aligned transversely relative to the flow of fluid medium.

19. A biological measurement apparatus as claimed in claim 18, wherein the first stimulus electrode is aligned with a flow of fluid medium only, without biological particles, and the second stimulus electrode is aligned with a flow of fluid medium and biological particles.

20. A biological measurement apparatus as claimed in any preceding claim, wherein the operative surfaces of the sensing electrodes of the first and second measurement capacitors are separated from each other, aligned transversely relative to the flow of fluid medium.

21. A biological measurement apparatus as claimed in claim 20, wherein the first stimulus electrode is aligned with a flow of fluid medium only, without biological particles, and the second stimulus electrode is aligned with a flow of fluid medium and biological particles.

22. A biological measurement apparatus as claimed in any preceding claim, wherein the sensing electrodes are each disposed adjacent to, but separated from, a corresponding stimulus electrode, thereby providing the first and second measurement capacitors.

23. A biological measurement apparatus as claimed in any preceding claim, wherein the distance between the electrodes of the first measurement capacitor and the electrodes of the second measurement capacitor is configured to be sufficient that the respective electrical fields are separated, such that a biological particle isn't detected by both measurement capacitors at the same time.

24. A biological measurement apparatus as claimed in any preceding claim, wherein a second complementary electrode arrangement is provided, which is disposed upstream or downstream of the electrode arrangement defined in any preceding claim, which constitutes the first electrode arrangement.

25. A biological measurement apparatus as claimed in claim 23, wherein the same electric stimulus signal is provided to the stimulus electrodes of each of the first and second electrode arrangements, with the stimulus electrodes of the first measurement capacitor in each arrangement being electrically connected to each other and the stimulus electrodes of the second measurement capacitor in each arrangement being electrically connected to each other.

26. A biological measurement apparatus as claimed in claim 25, wherein the stimulus electrodes of the first measurement capacitor in each arrangement are combined, and provided by a single stimulus electrode, and the stimulus electrodes of the second measurement capacitor in each arrangement are combined, and provided by a single stimulus electrode.

27. A biological measurement apparatus as claimed in claim 26, wherein the stimulus electrode of the first measurement capacitors of the electrode arrangements are disposed between the sensing electrodes of the first measurement capacitors of the electrode arrangements, and the stimulus electrode of the second measurement capacitors of the electrode arrangements are disposed between the sensing electrodes of the second measurement capacitors of the electrode arrangements.

28. A biological measurement apparatus as claimed in any one of claims 23 to 27, wherein the output signals from the sensing circuit associated with the first and second electrode arrangements are compared, such that a differential measurement is made.

29. A biological measurement apparatus as claimed in claim 28, wherein the concentration of biological particles and the flow rate of the fluid medium are controlled such that each biological particle in the fluid medium passes over one of the measurement capacitors of the first electrode arrangement and then, subsequently, passes over one of the measurement capacitors of the second electrode arrangement.
